SEQUENCE LISTING (11 sequences)

SEQ ID NO: 1 - DNA, 21 bp, Artificial Sequence
  primer up for base pair probe
ctggcctctctgattaaggag

SEQ ID NO: 2 - DNA, 21 bp, Artificial Sequence
  primer down for base pair probe
tcagatgatttcaggctttaa

SEQ ID NO: 3 - DNA, 22 bp, Artificial Sequence
  primer forward
tacagctgccaatgtctggatt

SEQ ID NO: 4 - DNA, 23 bp, Artificial Sequence
  primer reverse
cagtgtcccaggatttgtagaga

SEQ ID NO: 5 - DNA, 26 bp, Artificial Sequence
  probe
atcccaaaaataatcgccgctggcta

SEQ ID NO: 6 - DNA, 32 bp, Artificial Sequence
  forward primer
agcagaattccaggaggctgaagaagatctac

SEQ ID NO: 7 - DNA, 49 bp, Artificial Sequence
  reverse primer
ctcactcgagtcaggctttgaacttgcagacaaatgataattgggcatc

SEQ ID NO: 8 - DNA, 20 bp, Artificial Sequence
  forward primer
ttgtccagaaggttccaatg

SEQ ID NO: 9 - DNA, 20 bp, Artificial Sequence
  reverse primer
caaactcaggatacaagaaa

SEQ ID NO: 10 - DNA, 776 bp, Artificial Sequence
  rat gene coding sequence DNA
ccccccccaacagacttttgtctcagcctgcagagattgttgacttgcatcctaagcagaagacagtctgctgctcatcatgactcgcaacaaatatttcattctgctttcatgcctgatggtcctttctccaagccaaggccaggaggctgaagaagatctaccatctgccaggatcacttgtccagaaggttccaatgcctacagttcctactgttactacttcatggaagaccatttatcttgggctgaggcagatcttttttgccagaacatgaattcaggctacttggtgtcagtgctcagccaggctgagggcaactttctggcctctctgattaaggagagtggtactacagctgccaatgtctggattggcctccatgatcccaaaaataatcgccgctggcactggagcagtgggtctctgtttctctacaaatcctgggacactgggtatcctaacaattccaatcgtggctactgtgtatctgtgacttcaaactcaggatacaagaaatggagagataacagttgtgatgcccaattatcatttgtctgcaagttcaaagcctgaaatcatctgaaaaaaatagtcatacagagccagacaagaaaatactatggagtcaaaagtgaaactagaccatctatcaaaagcaaagtcaaccccctcttcctagacaaacattcttgcctcactgccctatggtatttttatctccattattgtatgtaaccctgcacatttaaataaaaataccttcacaataaaa

SEQ ID NO: 11 - protein, 165 aa, Artificial Sequence
  protein coding for rat
MTRNKYFILLSCLMVLSPSQGQEAEEDLPSARITCPEGSNAYSSYCYYFMEDHLSWAEADLFCQNMNSGYLVSVLSQAEGNFLASLIKESGTTAANVWIGLHDPKNNRRWHWSSGSLFLYKSWDTGYPNNSNRGYCVSVTSNSGYKKWRDNSCDAQLSFVCKFKA

(19) United States
(12) Patent Application Publication
(10) Pub. No.: US 2010/0099615 A1
(43) Pub. Date: Apr. 22, 2010

(54) PANCREATIC REGENERATING PROTEIN I IN CHRONIC PANCREATITIS AND AGING IMPLICATIONS FOR NEW THERAPEUTIC APPROACHES TO DIABETES

(75) Inventors: Martin H. Bluth, Southfield, MI (US); Michael Zenilman, Lawrence, NY (US)

Correspondence Address: HOFFMANN & BARON, LLP, 6900 JERICHO TURNPIKE, SYOSSET, NY, 11791, US

(73) Assignee: THE RESEARCH FOUNDATION OF STATE UNIVERSITY OF NEW YORK, ALBANY, NY (US)

(21) Appl. No.: 12/579,722

(22) Filed: Oct. 15, 2009

Related U.S. Application Data
(60) Provisional application No. 61/106,304, filed on Oct. 17, 2008.

(51) Int. Cl. A61K 38/17; C07K 14/435; C12P 21/02; A61P 3/10

(52) U.S. Cl. 514/12; 530/350; 435/69.1

(57) ABSTRACT

The present invention provides a method of treating diabetes, including administering to a mammal diagnosed with diabetes a purified recombinant reg I protein.

### CROSS REFERENCE TO RELATED PATENT APPLICATIONS

This application claims priority to provisional application 61/106,304, filed Oct. 17, 2008, which is herein incorporated by reference in its entirety.

### FUNDING STATEMENT

The present invention was made possible by an award from contract identifier R01 DK54511-01 awarded by the National Institute of Health. The government has certain rights to the invention.

### FIELD OF THE INVENTION

The present invention relates to the use of pancreatic regenerating protein I (reg I) as a treatment for diabetes. Specifically, the invention relates to methods for making pure recombinant reg I, methods, and uses thereof.

### BACKGROUND

Pancreatic regeneration protein is a component of pancreatic juice secreted by the pancreas. Roughly 15-16% of pancreatic juice includes pancreatic regeneration proteins. Through the years, there has been much research and discussion related to pancreatic regeneration protein, or Reg. As the research surrounding Reg has changed through the years, so too has the nomenclature. Reg may be referred to as pancreatic stone protein, pancreatic thread protein, peptide 23, and simply Reg. However, for the purposes of this research, it is important to distinguish that Reg, as well as its other common names, incorporates a larger family of proteins.

The pancreatic regeneration protein (Reg) family includes four known isoforms, including Reg I, Reg 2, Reg 3, and Reg 4 which vary in alternative nomenclature based on different species. The various family members of the Reg family do not have a complete overlap in homology. Rather, there is between 20-60% overlap on homology among the different Reg isoforms.

Over the years, researchers have conflicted in their results on whether Reg is or is not an indicator or characteristic of one or more functions and/or conditions in the body. Though Reg continues to be a subject of research, not much research has been done, to date in the area of using Reg as a treatment for diabetes in humans.

Throughout this patent application, reference will be made to publications providing methods and techniques known in the art. Each of the references cited herein is incorporated by reference in its entirety.

### SUMMARY OF THE INVENTION

An aspect of the present invention provides a method of treating diabetes, including administering to a mammal diagnosed with diabetes a purified recombinant reg I protein.

Another aspect of the present invention provides a purified recombinant pancreatic regeneration protein I (recombinant reg I), having the sequence ID: NM_{—}012641 (for rat) at about 100% homology. The human ID Number (genbank #) is NM_{—}006507 (FYI).

Still another example of the present invention includes a method of making a recombinant pancreatic regeneration protein I, including the steps of synthesizing the recombinant reg I protein; replicating the recombinant reg I protein; isolating the recombinant reg I protein; and purifying the recombinant reg I protein.

Still yet another aspect of the present invention provides a method of producing purified recombinant reg I, including: producing recombinant rat His-tagged reg I protein in E. coli through EcoRI-Xho I directional cloning; administering Xho/EcoRI restriction enzymes to amplify and digest a full-length reg I; inserting a plurality of digested reg I PCR amplicons in-frame into the pET24a bacterial expression vector to positively clone the reg I PCR amplicons; transforming the reg I PCR amplicons into BL21 (DE3) E. coli to grow; removing at least one soluble bacterial protein from the E. coli bacteria to obtain a bacterial pellet; washing and centrifuging the bacterial pellet to form a second bacterial pellet; resolubilizing the second bacterial pellet in a second resuspension buffer at low temperature; collecting the solubilized proteins onto a gel-bead; and spinning the beads and washing at least once with a wash buffer and centrifuged; administering an elution buffer to the beads; and dialyzing a reg I protein abundant elution in a dialysis buffer to enhance refolding and to prevent precipitation of the reg I protein.

### BRIEF DESCRIPTION OF THE DRAWINGS

FIG. 1. Hematoxylin and eosin staining of pancreas from 1 month (A) and 6 months (B) after PDL. Photomicrographs demonstrate neo-islet proliferation associated with ductal proliferation (arrow in A) and chronic atrophic pancreatitis with loss of acinar cells and ultimate dilation of ducts (asterisks in B; original magnification ×200).

FIG. 2. Pancreatic wet weight in animals 1 month (A), 6 months (B), and 12 months (C) after PDL insult compared with age-matched normal controls. Data are expressed as a ratio of pancreas wet weight over animal's total body weight (milligram per gram).

FIG. 3. The reg I mRNA quantitation (by Northern blot) In animals 1 month (A), 6 months (B), and 12 months (C) after PDL insult compared with age-matched normal controls. Data are expressed as arbitrary optical density (OD) units obtained from densitometry of the blots.

FIG. 4. Insulin mRNA quantitation (by Northern blot) in animals 1 month (A), 6 months (B), and 12 months (C) after PDL insult compared with age-matched normal controls. Data are expressed as arbitrary OD units obtained from densitometry of the blots.

FIG. 5. Glucose tolerance tests in animals 1 month (A), 6 months (B), and 12 months (C) after PDL insult±recombinant reg I treatment compared with age-matched BSA-treated controls. Data are expressed as milligram per deciliter serum glucose. Representative integrated area under the curve glucose responses for each time point (D). *P<0.05 of PDL animals compared with age-matched controls, \P<0.05 of untreated animals compared with 1-month controls, #P<0.05 of glucoses in reg I-treated animals compared with age-matched untreated PDL animals. C indicates control; P, PDL; R, recombinant reg I treatment.

FIG. 6. A, Real-time PCR analysis of reg I expression levels in pancreata obtained from 1-month-old and 12-month-old rats. B, Serum reg I levels when assessed in 1-month-old versus 12 month-old rats. Data are expressed as fold change and microgram per milliliter, respectively. C, Western blot analysis of reg I protein in pancreas obtained from normal 1-month-old and 12-month-old animals. Data represent 3 of 6 experiments with similar results. Pancreatic tissue lysates were processed blotted with monoclonal anti-reg I antibody as described in Materials and Methods. PJ indicates pancreatic juice.

FIG. 7. A, The GTTs in 2-, 12-, and 20-month-old rats. B, The GTT responses in 1-month-old rats in response to anti-reg I (anti-Reg) and nonspecific IgG (NS IgG) antibody treatment. Data are expressed as integrated area under the curve glucose responses (glucose× minutes). *P<0.05 for anti-reg I antibody treatment compared with untreated control animals.

FIG. 8. A, Representative integrated area under the curve glucose responses (glucose× minutes) for pre-reg I- and post-reg I-treated old (20-month-old) rats (n=6 per group); P=not significant. B, Representative IPGTT responses in old rats with elevated baseline IPGTT responses (>400 mg/dL, dark black line) in response to reg I treatment (light gray line); data represent 1 of 4 animals with similar results.

### DETAILED DESCRIPTION OF THE INVENTION

The research of the present inventors focuses on the relationship of pancreatic regenerating protein (reg I) in models of acinar cell atrophy and aging, and the effect of reg I protein replacement on glucose tolerance. Specifically, the inventors of the present invention have determined a method to isolate the Reg I protein and synthesize respectable yields of the reg I material to be used as a treatment for glucose intolerance and diabetes. The method is reproducible, with predicable yields. The present invention is not limited to methods of treatment of glucose intolerance and diabetes, and is understood to include treatment of any condition which may be aided through protein replacement.

Commercially available products are sometimes completely inactive or with reduced functionality. Further, though there is much research in and related to the pancreatic regeneration protein, few, if any, to date have published a reproducible synthesis with reasonable steps and a respectable yield. Thus, with the present invention, large quantities of the Reg I protein may be made through the batch synthesis and purification. As large quantities are synthesizable, the quantities may also be employed as treatments for various diagnoses, including for example, poor glucose tolerance testing, or diabetes, where glucose tolerance testing is one characteristic suggestive to a diabetic diagnosis.

Purified form, as used herein, generally refers to material which has been isolated under certain desirable conditions that reduce or eliminate unrelated materials, i.e. contaminants. Substantially free from contaminants generally refers to free from contaminants within analytical testing and administration of the material. Preferably, purified material is substantially free of contaminants is at least 50% pure, more preferably, at least 90% pure, and more preferably still at least 99% pure. Purity can be evaluated by conventional means, e.g. chromatography, gel electrophoresis, immunoassay, composition analysis, biological assay, NMR, and other methods known in the art.

The present invention may use recombinant Reg Ito treat mammals with pancreatic duct ligation for induced desirable glucose tolerance characteristics. In studies conducted herein, rats underwent pancreatic duct ligation (PDL) and were followed through 12 months. Aging rats were studied at 12 and 20 months. Intraperitoneal glucose tolerance tests (IPGTTs) were performed, pancreatic reg I, reg I receptor, insulin gene expression, and reg I protein levels were measured. Pancreatic duct ligation and aged animals were treated with exogenous reg I protein and assessed for glucose metabolism.

The present invention is applicable to various subjects, and is not limited to those subjects directly studied herein. The term “subject”, as used herein may refer to a patient or patient population diagnosed with, or at risk of developing the conditions described herein. Also, as used herein, a subject may refer to a living animal, including mammals, which may be treated with the methods and compounds of the present invention or which need treatment. Such subjects may include mammals, for example, laboratory animals, such as mice, rats, and other rodents; monkeys, baboons, and other primates, etc. They may also include household pets or other animals in need of treatments. In addition, the subjects may include mammals such as humans.

The present invention may be administered to a subject in an amount effective in achieving its purpose. The effective amount of the material to be administered can be readily determined by those skilled in the art, for example, during pre-clinical trials and clinical trials, by methods familiar to physicians and clinicians. An effective amount of a material useful in the methods of the present invention, preferably in a pharmaceutical composition, may be administered to a mammal in need thereof by any of a number of well-known methods for administering pharmaceutical compounds. The material may be administered systemically or locally.

Any formulation known in the art of pharmacy is suitable for administration of the materials useful in the methods of the present invention. For oral administration, liquid or solid formulations may be used. Some examples of formulations include tablets, capsules, such as gelatin capsules, pills, troches, elixirs, suspensions, syrups, wafers, chewing gum and the like. The materials can be mixed with a suitable pharmaceutical carrier (vehicle) or excipient as understood by practitioners in the art. Examples of carriers and excipients include starch, milk, sugar, certain types of clay, gelatin, lactic acid, stearic acid or salts thereof, including magnesium or calcium stearate, talc, vegetable fats or oils, gums and glycols.

Formulations of the materials useful in the methods of the present inventions may utilize conventional diluents, carriers, or excipients etc., such as those known in the art to deliver the materials. In some embodiments the formulation will include a material suitable to protect the material from being destroyed in the body, for example, in the stomach of the subject. For example, the formulations may comprise one or more of the following: a stabilizer, a surfactant, preferably a nonionic surfactant, and optionally a salt and/or a buffering agent. The material may be delivered in the form of an aqueous solution, or in a lyophilized form. Similarly, salts or buffering agents may be used with the compound.

The present invention may be administered in therapeutically effective concentrations, to be provided to a subject in standard formulations, and may include any pharmaceutically acceptable additives, such as excipients, lubricants, diluents, flavorants, colorants, buffers, and disintegrants. Standard formulations are well known in the art. See, e.g. Remington's pharmaceutical Sciences, 20th edition, Mach Publishing Company, 2000. The formulation may be produced in useful dosage units for administration by any route that will permit the material to enter the bloodstream and perform its desired function. Exemplary routes of administration include oral, parenteral, transmucosal, intranasal, insulfation, or transdermal routes. Parenteral routes include intravenous, intra-arterial, intramuscular, intradermal, subcutaneous, intraperitoneal, intraductal, intraventricular, intrathecal, and intracranial administrations.

The pharmaceutical forms suitable for injection include sterile aqueous solutions or dispersions and sterile powders for the extemporaneous preparation of sterile injectable solutions or dispersions. The ultimate solution form in all cases should be sterile and fluid. Typical carriers include a solvent or dispersion medium containing, e.g., water buffered aqueous solutions, i.e., biocompatible buffers, ethanol, polyols such as glycerol, propylene glycol, polyethylene glycol, suitable mixtures thereof, surfactants or vegetable oils. Sterilization may be accomplished utilizing any art-recognized technique, including but not limited to filtration or addition of antibacterial or antifungal agents.

The materials of the present invention may be administered as a solid or liquid oral dosage form, e.g. tablet, capsule, or liquid preparation. The materials may also be administered by injection, as a bolus injection or as a continuous infusion. The materials may also be administered as a depot preparation, as by implantation or by intramuscular injection.

The materials referenced in the present invention may be in a “pharmaceutically acceptable carrier”. A pharmaceutically acceptable carrier includes any and all solvents, dispersion media, coatings, antibacterial and antifungal agents, isotonic agents and the like. The use of such media and agents are well-known in the art. The phase ‘pharmaceutically acceptable’ refers to molecular entities and compositions that are physiologically tolerable and do not typically produce unwanted reactions when administered to a subject, particularly humans and other mammals. Preferably, as used herein, the term “pharmaceutically acceptable” means approved by a regulatory agency of the federal or a state government or listed in the U.S. Pharmacopeia or other generally recognized pharmacopeia for use in animals, and more particularly, in humans. The term carrier refers to a diluent, adjuvant, excipient or vehicle with which he compounds may be administered to facilitate delivery. Such pharmaceutical carriers can be sterile liquids, such as water and oils, or organic compounds. Water or aqueous solution saline solutions, and aqueous dextrose and glycerol solutions are preferably employed as carriers, particularly as injectable solutions.

The experiments related to the present invention showed the following results. After PDL, chronic atrophic pancreatitis developed, with a progressive loss of acinar cells and pancreatic reg I. During aging, a similar depression of reg I gene expression was also noted. The reg I levels correlated with pancreatic insulin levels. Twelve months after PDL, IPGTT results were abnormal, which were significantly improved by administration of reg I protein. Aged animals demonstrated depressed IPGTT, which marginally improved after reg I administration. Anti-reg antibody administration to young rats depressed IPGTT to elderly levels.

Thus, depletion of the acinar product reg I is associated with the pathogenesis of impaired glucose tolerance of pancreatic diabetes and aging, and replacement therapy could be useful in these patients. Such replacement would be done by administering to the mammal, here, a rat, an amount of recombinant reg I.

The mass of the exocrine pancreas diminishes with time in both chronic pancreatitis and aging.¹ Both processes are associated with glucose intolerance and ultimately clinical diabetes. Pancreatic (Sandmeyer) diabetes occurs in 40% to 60% of patients with chronic pancreatitis; its development correlates with progressive destruction of acinar cells. The loss of an acinar cell factor might play a role in its progression.² Diabetes is a disease of aging; greater than 20% of patients over the age of 80 years will develop it³ and also may be linked to acinar cell loss.

Regenerating protein I (reg I) is a product of the acinar cells of the pancreas, and its genetic expression is linked to β-cell function. Its gene is induced during ductal proliferation, β-cell growth, and islet regeneration.^{4,5} The reg I protein is mitogenic to ductal and β cells,⁵⁻⁷ and its administration after islet failure reverses diabetes.^{8,9} The gene for the receptor of reg I has been isolated,¹⁰ and was shown to be involved in the differentiation of the exocrine pancreatic cells.¹¹

It has been postulated that in chronic pancreatitis and aging, pancreatic exocrine reserves of reg I are progressively depleted, and this depletion leads to islet failure, glucose intolerance, and diabetes. To test this hypothesis, three models were used. For chronic pancreatitis, the model of pancreatic duct ligation (PDL) was used, which induces acinar cell atrophy. Using a technique modified from Edstrom et al,¹²⁻¹⁵ only acinar cells are affected; islets remain functional until after the acinar cells atrophy and glucose intolerance develops.^{16,17} Also, a model of longitudinal aging in normal rats was used. Finally a model of “induced aging” was tested by administering antibodies to reg Ito young rats.

In all studies, glucose tolerance was measured by intraperitoneal glucose tolerance tests (IPGTTs), reg I and receptor gene expression, insulin levels, and serum reg I levels. The studies were conducted to determine whether administration of a recombinant reg I protein could improve glucose tolerance in animals with impaired IPGTT.

### MATERIALS AND METHODS

### Modified Subtotal PDL Model of Chronic Pancreatitis

The major and minor pancreatic ducts of 6- to 8-week old 150-g female Wistar rats were ligated as follows: after 50 mg/kg Nembutal anesthesia, a midline laparotomy was performed. Modified subtotal ligation of the pancreas ¹³⁻¹⁷ was accomplished by initially dissecting, ligating, and dividing the main ducts to the splenic and gastric lobes. Using microscopic dissection, the duodenal and parabiliary lobes were disconnected from the duodenum and common bile duct, thereby detaching the pancreas off these structures.

Animals were analyzed by IPGTT, reg I and receptor gene expression and serum reg I protein levels at 3 time points after PDLV1, 6, and 12 months. After IPGTT, the animals were recovered, and the next morning, animals were killed by asphyxiation. The protocol was approved by the Animal Care and Use Committee.

### Pancreatic Wet Weight

Pancreatic wet weight as a marker for tissue edema was quantitated by the ratio of pancreas wet weight over the animal's total (milligram per gram) body weight.¹⁸

### Intraperitoneal Glucose Tolerance Testing

Intraperitoneal glucose tolerance tests were performed under Nembutal anesthesia (intraperitoneal injection of 50 mg/kg rat body weight). Glucose was measured by orbital or tail vein bleed, at 15- to 30-minute intervals after intraperitoneal injection of glucose (1 g/kg), by glucose oxidase (Beckman Instruments) or by amperometry (Accu-Check Advantage, Roche Diagnostics) according to manufacturer's instructions.

### Insulin and Reg I Measurements

Serum insulin was measured at the indicated time points by enzyme-linked immunosorbent assay (Crystal Chem, Inc, Downers Grove, Ill.) according to the manufacturer's instructions. Serum levels of reg I protein concentrations were determined by direct enzyme-linked immunosorbent assay in a manner described previously¹⁹ using a monoclonal antibody to reg I.²⁰

### Northern Blot Analysis

A 202-base pair probe for rat reg I was produced by reverse transcription-polymerase chain reaction (RT-PCR) from rat pancreatic RNA using primers (up: 5′-CTG GCCTCTCTGATTAAGGAG-3′ [Seq. ID No. 1], down: 5′-TCAGATGATTT CAGGCTTTAA-3′ [Seq. ID No. 2]).²¹ This sequence is homologous to the mouse reg I published by Unno and colleagues,²¹ and is within the rat reg I family, unique to reg I. The size of the PCR product was confirmed by electrophoresis. The PCR product was then ultrafiltered using a 30,000-molecular weight filter (Millipore, Bedford, Mass.) to remove unincorporated dNTPs. The reg I receptor complementary DNA (cDNA) was prepared by double digestion of pClneo-reg I receptor cDNA plasmid¹⁰ with HindIII and Not I. Electrophoresis of the digestion complex was performed on a 0.8% agarose gel, after which the receptor band was cut from the gel, and the cDNA was extracted using the QIAEX II Agarose Gel Extraction protocol (Qiagen, Germany). Probe DNA was labeled for chemiluminescent imaging with DIG High Prime DNA Labeling and Detection Kit (Boehringer Mannheim, Roche Diagnostics, Indianapolis, Ind.). A probe for rat insulin-I was a gift from Luciano Rossetti (Department of Medicine, Albert Einstein College of Medicine) and was similarly labeled.

Total pancreatic RNA was isolated by the TRIREAGENT technique. Ten micrograms of total RNA was analyzed by 1% formaldehyde-agarose gel electrophoresis to document integrity. RNA was transferred to nitrocellulose filters and analyzed by standard Northern blot. To correct for loading, the blots were stripped and reprobed with digoxygenin-labeled oligo-dT and quantitated using NIHImage (Scion Corp, Frederick, Md.). Data are expressed as corrected counts (OD reg/OD oligo-dT) after background subtraction and reported as mean T SEM. Statistical analysis was performed by unpaired Student t tests, and significance was defined as P<0.05.

### Real-Time Quantitative RT-PCR

One-step real-time quantitative RT-PCR for reg I messenger RNA (mRNA) was performed as previously described²² using a GeneAmp 5700 sequence-detection system (Applied Biosystems, Foster City, Calif.), with A-actin as an endogenous control to standardize the amount of sample RNA added to a reaction. Primers and probes were designed using Primer Express software (Applied Biosystems); the specific forward and reverse primers were designed based on published sequences of rat reg I (GenBank accession no. NM_{—}012641). All primers and probes and other reagents for real-time quantitative PCR were purchased from Applied Biosystems (forward: 5′-TACAGCTGCCAATGTCTGGATT-3′ [Seq. ID No. 3], reverse: 5′-CAGTGTCCCAGGATTTGTAGAGA-3′ [Seq. ID No. 4], probe: 5′-FAM-ATCCCAAAAATAATCGCCGCTGGC-TA-3′ [Seq. ID No. 5]). One hundred nanograms of total RNA was used to set up 25-μL real-time quantitative PCRs that consisted of 1× TaqMan Universal PCR Master Mix, 500 nM forward and reverse primers, and 200 nM TaqMan probe. The PCR amplification was carried out with the following temperature profile: 30 minutes at 48° C., 10 minutes at 95° C., and 40 cycles of 15 seconds at 95° C. and 1 minute at 60° C. Assays were performed in triplicate. Data were analyzed with the relative standard curve method. Standard curves of the genes of interest and A-actin were prepared with three 1:2 dilutions (4 points, 8-fold range) of total RNA from one of the samples that was expected to have the highest amount of mRNA for the gene of interest. For each reaction tube, the amount of target or endogenous reference was determined from the standard curves. The mean amount of each sample was calculated from the triplicate data and was normalized by division by the mean quantity of β-actin RNA for the same sample. The mean and SD of each treated group were calculated from the normalized value for each rat in that group.

### Isolation/Production of Reg I Protein

Recombinant rat His-tagged reg I protein was produced in Escherichia coli through EcoRI-Xho I directional cloning (Forward primer: 5′-AGCAGAATTCCAGGAGGCTGAA GAAGATCTAC-3′ [Seq. ID No. 6]; reverse primer: 5′-CTCACTCGAGT CAGGCTTTGAACTTGCAGACAAATGATAATTGGG CATC-3′ [Seq. ID No. 7]). Full-length reg I was PCR amplified and digested with Xho/EcoRI restriction enzymes. The reg I-containing constructs were confirmed by PCR (forward: 5′-TTGTCCA GAAGGTTCCAATG-3′ [Seq. ID No. 8], reverse: 5′-CAAACTCAGGATA CAAGAAA-3′ [Seq. ID No. 9]). Digested reg I PCR amplicons were inserted in-frame into the pET24a bacterial expression vector (Novagen, San Diego, Calif.). Positive clones were transformed into BL21 (DE3) E. coli, grown to a density of 2.0 OD in 500 mL of TB broth with kanamycin and induced for 3 hours at 37° C. with 2 mM isopropyl-beta-d-thiogalactopyranoside. The bacteria was centrifuged and resuspended in resuspension buffer (0.1 M sodium phosphate, pH 8.0, with 1 mM phenylmethanesulfonyl fluoride and 1 mM dithiothreitol) containing protease inhibitors and sonicated on ice. Soluble bacterial proteins were disposed, and the bacterial pellet was sequentially washed first with wash buffer A (1.5% triton, 0.1 M sodium phosphate, 1 mM phenylmethanesulfonyl fluoride) and centrifuged at 12,000 revolutions per minute for 10 minutes, and then with wash buffer B (0.5% triton, 0.1 M sodium phosphate) and pelleted. Because reg I formed inclusion bodies, the bacterial pellet was resolubilized in 15 mL of resuspension buffer (6 M urea, 0.1 M sodium phosphate, pH 8.0) for 10 minutes on ice. Solubilized proteins were collected after centrifugation at 4° C. for 10 minutes at 10,800 revolutions per minute and batch bound to prepared His-Select Nickel Affinity Gel beads end-over-end overnight at 4° C. The next day, the beads were spun down and washed 5 times with wash buffer (0.1 M sodium phosphate, pH 7.0, 6 M urea) and centrifuged. Five milliliters of elution buffer (0.1 M sodium phosphate, pH 4.5, 6 M urea) was added to the beads and batch eluted end-over-end for 4 hours or overnight at 4° C. 3 times. Samples of all washes, eluates, and the beads at each step were analyzed by sodium dodecyl sulfate-polyacrylamide gel electrophoresis. Eluates containing abundant reg I protein were dialyzed in dialysis buffer (0.1 M sodium phosphate, 50 mM acetic acid, pH 4.5) in stepwise fashion, with decreasing amounts of urea (from 6 M urea down to no urea) to enhance refolding and to prevent precipitation of the reg protein over several hours. Protein concentrations were determined by the Bradford protein assay and confirmatory sodium dodecyl sulfate-polyacrylamide gel electrophoresis Coomassie stain.

The yields for the final purified recombinant reg I were consistently around 20%, with about 10% fluctuation compared with the crude starting material as determined by western blot.

### Reg I Treatment (in PDL Experiment)

Two weeks before the study, animals (n=6 per group) were injected with either 0.1 mg/mL per day of bovine serum albumin (BSA) or 1 mg/kg per day of recombinant rat reg I protein.⁸

### Aging Study

Twenty-month-old female Wistar rats (n=12) underwent baseline IPGTTs. The IPGTT curves were similarly established for ten 1-month-old (n=10) and 12-month-old (n=6) female Wistar rats for reference. Twenty-month-old rats were then randomly divided into 2 groups (n=6) and injected intraperitoneally with either recombinant rat reg I protein (1 mg/kg per day)²³ or vehicle (50 mM acetic acid, 0.1 M sodium phosphate, pH 4.5, 1 mg/mL BSA) for a period of 14 days, at which time IPGTT curves were again determined and compared with the baseline curves. A subset of reg I-treated animals was again tested 14 days later.

### Anti-Reg I Antibody Administration

Young rats were treated with mouse anti-human reg I monoclonal antibody²⁰ (2.5 mg/kg) intravenously (internal jugular vein) via osmotic pumps for 7 days. Control antibody treatment consisted of nonspecific mouse immunoglobulin G (IgG). The IPGTT measurements were taken before and after treatment.

### Statistics

Animals were compared by unpaired Student t tests. Glucose kinetics (integrated areas of glucose) were compared longitudinally to reg I levels (serum protein and pancreatic mRNA) by correlation coefficient analyses. Pretreatment and posttreatment IPGTT curves were compared at each time point and statistically analyzed using the Wilcoxon signed rank test and Student paired t tests. For all analyses, statistical significance was defined as P<0.05.

### Results

### Studies on Chronic Atrophic Pancreatitis Effect of PDL on Pancreatic Wet Weight

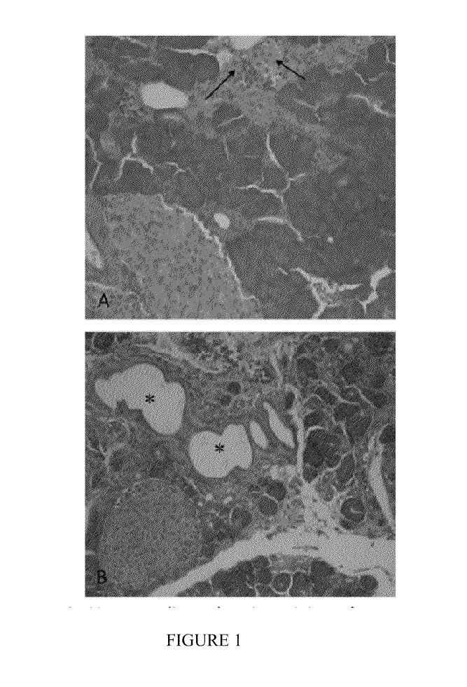

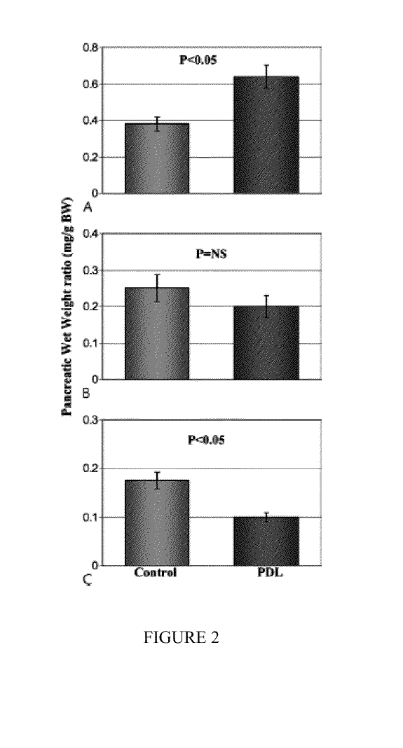

After PDL, as shown in FIG. 1, chronic atrophic pancreatitis was noted, with loss of acinar cells and hyperplasia of ducts and islets. In FIG. 2A, 1 month after PDL, there was a statistically significant increase in pancreatic wet weight when compared with controls. Six months post-PDL, there were no observable differences in wet weights from control animals (FIG. 2B), but at 12 months, animals who underwent PDL demonstrated decreased pancreatic wet weights (FIG. 2C). When PDL-insulted animals were treated with exogenous recombinant reg I (see later), no observable differences in pancreatic wet weight were noted at 6 and 12 months post treatment when compared with PDL-insulted control (BSA-treated) animals (data not shown; 1-month reg I treatment after PDL not tested).

### Effect of PDL on Reg I, Insulin, and Reg I Receptor Expression

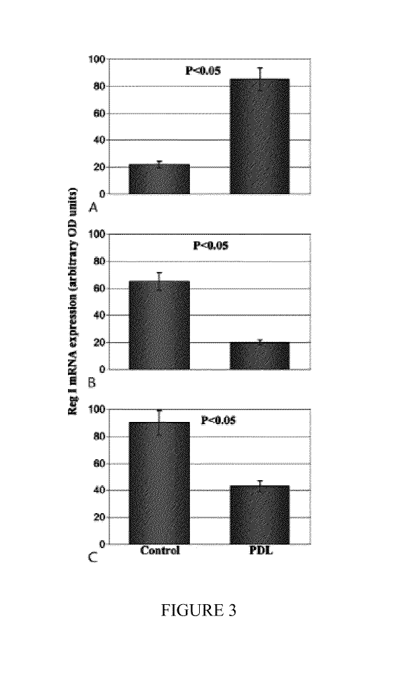

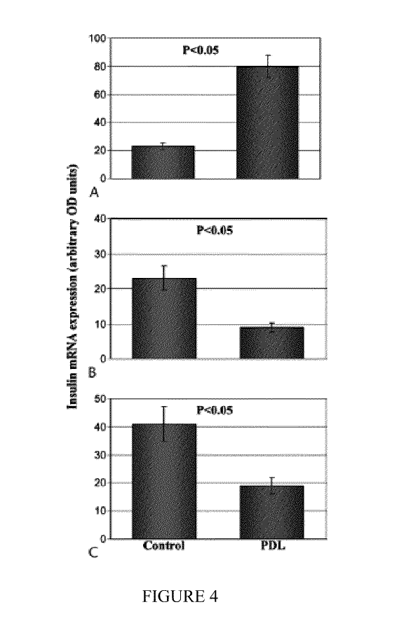

One month after animals were insulted with PDL, reg I gene expression increased when compared with age-matched control animals (FIG. 3A). In contrast, reg I expression was found to decrease at 6 (FIG. 3B) and 12 months (FIG. 3C) after PDL. Western blot analysis of protein levels showed similar results (data not shown). Pancreatic insulin mRNA levels paralleled reg I levels; they were high at 1 month and depressed at 6 and 12 months post-PDL (FIGS. 4A-C). This correlation of expression of reg Ito insulin was statistically significant (r=0.83, P<0.0001).

Serum levels of reg I did not change between groups (data not shown), and although serum insulin levels increased mildly at 1 month, they were not statistically different from controls at 12 months after PDL (data not shown). No significant differences in reg I receptor expression were observed at 1 and 12 months after PDL compared with controls (data not shown).

### Effect of PDL on Serum Glucose

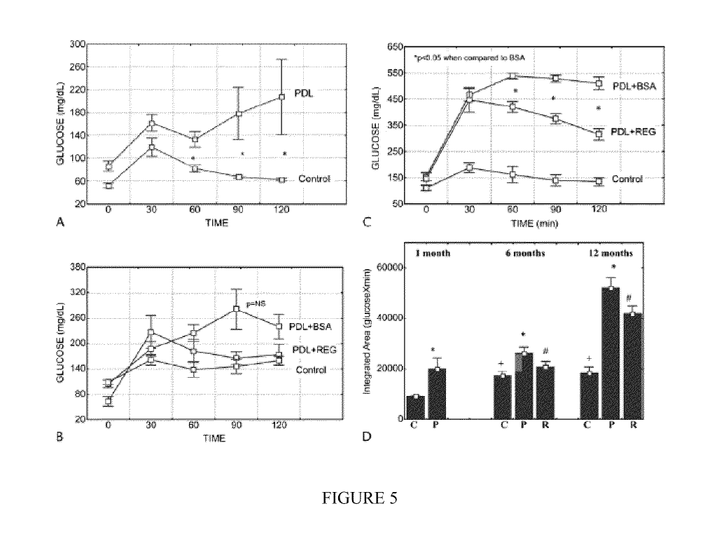

Glucose tolerance in the PDL-treated animals was assessed by IPGTT and compared them with age-matched normal controls. FIG. 5 demonstrates that IPGTT responses worsened with normal aging (see controls in FIGS. 5A, B, and C, respectively, and the integrated areas depicted in FIG. 5D). FIG. 5 also shows that IPGTTs were worse at 1, 6, and 12 months after PDL insult when compared with control animals (FIGS. 5A-C). Integrated areas for all experiments are shown in FIG. 5D.

### Reg I Protein Treatment

Because abnormal IPGTT correlated with depressed reg I levels, it was therefore postulated that replacement of reg I protein by intraperitoneal injections would improve IPGTT responses at 6 and 12 months when compared with PDL alone (FIGS. 5B, C). Age-matched animals were treated with 1 mg/kg recombinant reg I or BSA for 2 weeks and subjected to IPGTT.

No effect was observed in 1-month or 6-month PDL animals. However, FIG. 5C shows that treatment of 12-month PDL animals with reg I protein resulted in a statistically significant improvement in glucose tolerance. FIG. 5D shows integrated glucose responses, demonstrating worsening responses with PDL and improvement with reg I treatment in the 12-month PDL animals alone.

The reg I protein treatment had no effect on pancreatic wet weight pancreatic mRNA expression of reg I, insulin or reg I receptor, or serum levels of reg I or insulin (data not shown).

### Studies on Aging

### Effect of Aging on Reg I Expression and Its Relationship to Glucose Metabolism

The observation of age-related depression of reg I gene expression, age-related impaired IPGTT, and partial reversal of impaired GTT with reg I treatment in acinar cell-depleted rats led us to further investigate the relationship of reg I, aging, and diabetes. Of particular interest was the role of reg I as therapy for diabetes.

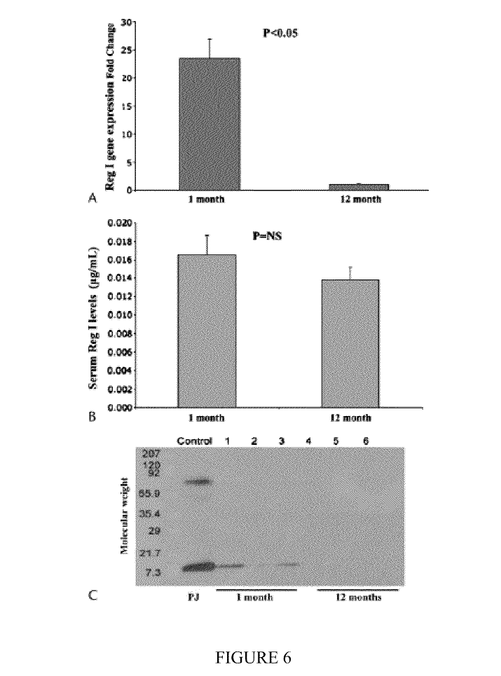

Pancreatic reg I gene expression in aged animals was first studied by real-time PCR, then protein by Western blot analysis. As shown in FIGS. 6A and C, pancreata obtained from 1-month-old rats had elevated levels of reg I mRNA and protein (FIG. 6C) expression when compared with 12-month-old rats (<20-fold increase in mRNA expression, P<0.05). This depression persisted at the same level until 20 months (data not shown). Interestingly, levels of serum reg I protein did not differ between groups (FIG. 6B).

### Induction of Aging by Reg I Antibody

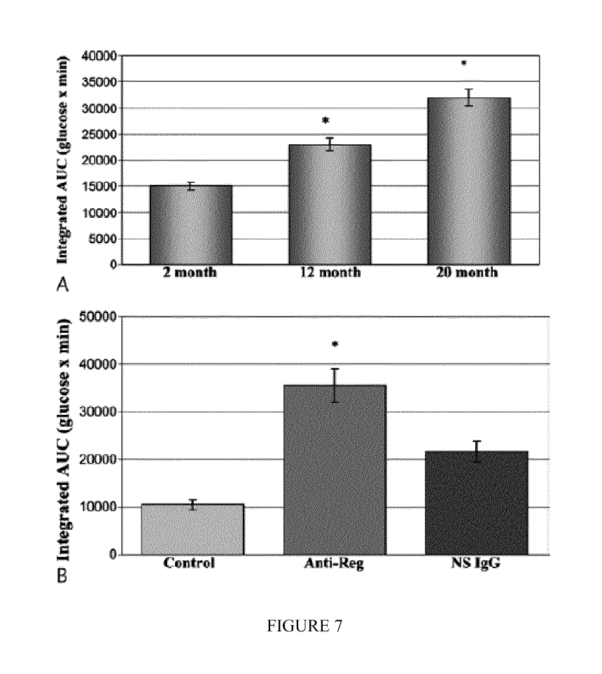

FIG. 7A demonstrates that compared with young rats, older rats have developed impaired IPGTT. Remarkably, when young rats were chronically infused with anti-reg I antibody for 1 week, worsening of IPGTT responses was observed when compared with untreated animals and controls (nonspecific IgG injection; FIG. 7B). The level of impairment (as measured by integrated areas under the curve) induced by reg I antibody in 1-month-old rats approached the level of 20-month-old rats.

### Effect of Recombinant Reg I Treatment and Glucose Metabolism

The effect of recombinant reg I treatment, compared with vehicle, in 20-month-old animals, was then investigated. Baseline fasting glucose measurements obtained from 2-month-old animals displayed lower basal glucose levels when compared with 12- or 20-month-old animals (77±3 mg/dL, 92±3 mg/dL, 91±4 mg/dL, respectively; P<0.05). Twenty month-old rats treated with recombinant reg I protein drastically improved fasting glucose levels compared with pretreated rats (79±3 mg/dL vs. 91±4 mg/dL, P<0.05) and approached values obtained from 2-month-old rats.

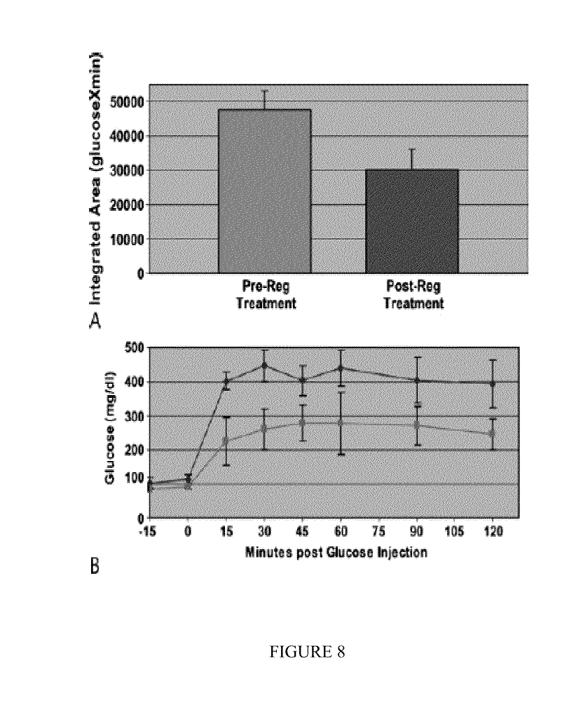

The effect of recombinant reg I treatment, compared with vehicle, in 20-month-old animals, on impaired IPGTT, was then investigated. Although there was no statistical difference between the mean±SEM integrated areas under the curve between recombinant reg I- or vehicle-treated animals (P=not significant; FIG. 8A), it was found that 4 of the 6 older animals who had 4 or more time point measurements that were within or higher than the upper 95% confidence interval above normal did show some improvement (FIG. 8B; P=0.07). There were no differences in serum insulin levels when 20-month-old rats were treated with recombinant reg I protein when compared with untreated control animals (data not shown). But treatment of 20-month-old rats with recombinant reg I protein induced increased expression of reg I mRNA in pancreas (n=2, data not shown), suggesting a positive feedback loop of protein production.

### Discussion of Test Data

The relationship between the exocrine and endocrine pancreatic mass has been separately studied for years. Although integrated structurally, there is a paucity of data regarding their functional relationship. But as the exocrine pancreas atrophies—either by disease or aging—the endocrine pancreas shows signs of failure. By exploring this relationship, it is believed that pancreatic reg I is the link.

Insulin-dependant diabetes mellitus is a late feature of chronic pancreatitis and has been called Sandmeyer diabetes. Although it occurs after loss of 70% to 80% of the islet mass, its cause has eluded investigators. To date, the only explanation given for this progressive β-cell failure is that the severe fibrotic degeneration of acinar tissue—“acinar sclerosis”—eventually chokes the islet of local circulation and glucose diffusion. This theory has never been proven.^{24,25} It is also likely that the loss of other substances from the acinar pancreas is involved in this islet cell failure.^{2,26}

The pancreatic glandular tissue atrophies with age in a manner that is discernible in humans on computed tomographic scan. By the age of 85 years, the gland has lost one third its weight, and histology shows replacement of parenchyma by fatty infiltration and fibrosis. ^{1,27} Along with pancreatic atrophy, the width of the main pancreatic duct increases at a rate of 8% per decade, with occasional ductal proliferation and metaplasia.

Although functional changes in the exocrine pancreas during aging are clinically barely noted, changes in the endocrine pancreas can be noticeable. Only 3% of persons aged 18 to 24 years have mild glucose intolerance, but the incidence is as high as 42% in persons aged 75 to 79 years. Similarly, 16% of the population over the age of 80 years is clinically diabetic.³

Evidence that the acinar cell plays a critical role in islet β-cell development and maintenance is very strong. In experimental models of chronic atrophic pancreatitis, progressive loss of islet function parallels the loss of acinar tissue. For instance, after ligation and division of the rat main pancreatic duct, islets progressively lose their regenerative capacity and involute, paralleling the atrophy of the surrounding exocrine (acinar) tissue.^{15,28,29} Similarly, PDL in the dog leads to progressive exocrine atrophy and islet failure.^{24,30} Histological analysis of these islets has demonstrated progressive loss of β-cell mass,³¹ and physiological studies show progressively diminished insulin secretion capacity if the pancreatic duct was ligated and not internally drained, paralleling progressive exocrine failure.

The role of pancreatic reg I in islet function is of particular interest. It is an acinar product that has been shown to modulate islet function. The reg I mRNA is constitutively expressed in acinar cells, its expression parallels islet gene expression,^{6,32} and its gene is induced before and during islet regeneration.^{33,34} Furthermore, reg I gene expression has been directly linked to insulin gene expression.^{35,36} Patients who harbor antibodies to reg I have developed diabetes,³⁷ and reg-knockout mice show poor β-cell recovery and regeneration after insult.³⁸ The reg administration showed that amelioration of surgical-induced (depancreatized) diabetes⁸ and transgenic overexpression of reg in islets is linked to the development of tumors.³⁹ Reg I proteins are mitogenic to pancreatic-derived cell lines ARIP (ductal) and RIN (β cell),⁴ and to isolated pancreatic ducts in culture,⁷ and likely exert their effect via the mitogen-activated protein kinase P38 pathway.⁴⁰ The rat reg I receptor¹⁰ has recently been cloned and is a transmembrane 919-amino acid protein. Cells that express the receptor proliferate in response to reg I protein.¹⁰ The present invention demonstrates that the receptor gene is induced along with reg I after pancreatitis.^{6,41}

The potential for reg I protein as a treatment of diabetes has been proposed by showing that exogenous administration of recombinant rat reg I protein can reverse diabetes after massive pancreatic resection, and it is mitogenic to B cells within the islet.

The observation that reg I gene expression correlates with islet proliferation and gene expression⁴² supports the hypothesis that this factor, from the exocrine pancreas, is involved in maintaining islet β-cell integrity. To date, reg I is the only islet growth factor known to be directly derived from the acinar cell. It could exert its effect by endocrine or paracrine actions.

A homologue of reg I, islet neogenesis-associated protein, has been isolated from regenerating pancreata,^{43,44} which, similar to reg I, promotes islet regeneration. A bioactive islet neogenesis-associated protein fragment has been identified,⁴⁵ which also promotes β-cell growth, PDX gene expression, and has reversed diabetes in mice. Similarly, a bioactive fragment in a homologous region of reg I has been identified,⁴⁰ which confers mitogenesis to ductal and B cells; but exogenous administration had no effect in any current models of acinar failure associated with impaired glucose tolerance (Bluth et al.).

It is believed that reg I treatment would increase β-cell mass, as has been shown in vitro and others in vivo.⁸ The islet mass was not measured herein, and measurement of total pancreatic BrdU incorporation by Southern blot did not show an increase (data not shown). But Watanabe and colleagues⁸ did show clear islet-specific BrdU incorporation after reg I treatment. Aside from β-cell expansion, other factors such as the glucose sensitivity of islets, peripheral utilization of glucose, or insulin receptor sensitivity can be involved. But other studies suggest that this is unlikely—no effect of reg I insulin secretion and sensitivity to glucagon-like peptide (GLP-1) has been seen. In fact, preliminary studies on host insulin sensitivity by intravenous insulin tolerance showed no effect by reg I.

First, it was studied whether reg I can be involved with Sandmeyer diabetes using a model of chronic atrophic pancreatitis, as induced by modified subtotal ductal ligation in the rat. After duct ligation, animals did not appear ill and, in fact, gained weight. It was observed that acinar cells alone are affected, ducts are preserved, and islets are unaffected until after the acinar cells atrophy.¹²⁻¹⁷ In this model, pancreatic wet weight, a marker of edema, was initially increased at 1 month and then decreased at 12 months post-PDL. It is likely that the PDL model initially mimics pancreatitis, explaining the initial increase in pancreatic edema.

Gene expression patterns for both reg I and insulin correlates significantly, an observation that has previously observed. The reg I and insulin initially increased at 1 month after PDL, perhaps as a result of pancreatitis, but at 6 months and 1 year, as the acinar cells involute, both decrease. Glucose metabolism, as measured by IPGTT, gradually became more impaired over the year. Although the originally published experiments with ductal ligation of the splenic lobe alone gave inconsistent glucose intolerance, the modification of disconnecting the pancreas from the common bile duct yielded persistent glucose intolerance at 6 and 12 months postoperatively. The worst IPGTTs were at 1 year, when reg I and insulin were depressed the most compared with those of the controls. Administration of recombinant reg I protein improved IPGTT at 12 months.

It is therefore likely that reg I can affect glucose control in chronic pancreatitis and may be a useful therapeutic modality. Although the reg I peptide used herein was bioactive, 40 studies using it in this model failed—the intact protein is critical for the effect.

Further experiments also showed in the normal aging rat that reg I levels decrease in parallel to insulin, as IPGTT gradually becomes impaired. Using this information, a longitudinal study of reg I and IPGTT in aging was developed. It was noted that as animals aged to 20 months, reg I gene expression decreased, and their glucose tolerance became impaired. In concert with these findings, in normal young rats, treatment with a monoclonal antibody to reg I induced IPGTT similar to that of an old one, suggesting a direct effect of reg I on glucose metabolism and may be age dependent.

Finally, it was postulated that reg I was involved in abnormal glucose tolerance in the aging pancreas, and replacement therapy would improve tolerance. Such postulating is based on preliminary data where it was demonstrated that aged rats that were treated with recombinant reg protein had decreased levels of glycohemoglobin and hemoglobin A1C when compared with pretreated animals, where these analytes were used as a marker of glucose control.⁴⁶ Significant improvement in glucose tolerance in older PDL animals was observed, which were treated with reg I protein. However, no effect of reg I treatment on IPGTT responses in older animals as a group was observed. In normal older animals with impaired baseline IPGTT, an interesting trend toward improved glucose tolerance was noted after recombinant reg I treatment. It is likely that the PDL-insulted animals had more significant loss of acinar cells than normal aged ones, and that reg I treatment may serve as an ideal therapy in the setting only of severe glucose intolerance specifically associated with severe acinar cell loss. It is also possible that the dose, number of animals used, or duration of reg I therapy used in the current studies was insufficient to demonstrate the desired effect.

In rats with severe acinar depletion (PDL), reg I treatment yields a partial reversal of impaired IPGTT. In aged rats with less acinar cell loss but depressed reg I levels and impaired IPGTT, some improvement may occur. The partial responses to reg I therapy may be because the dose used was suboptimal. More frequent administration of recombinant reg I or increased concentration per dose might demonstrate significant improvement of glucose tolerance in both models. High-scale production of recombinant reg I protein would be necessary to produce sufficient quantities of reg I for studies in rats and higher-order vertebrates to determine appropriate route and dosing protocols for maximal therapeutic potential.

In conclusion, the data shows that progressive loss of the pancreatic acinar cell is directly related to the development of glucose intolerance, and that reduced pancreatic reg I may be responsible for this effect. Progressive islet failure in chronic pancreatitis is likely not the result of islet sclerosis, but secondary to the loss of functional acinar cells and pancreatic reg I. Progressive islet failure of aging is the result of a progressive loss of exocrine cells, which harbor the reg I protein. Pancreatic reg I is therefore an acinar product that appears to directly affect glucose tolerance, possibly through an effect on the islet (B cell). Future studies are needed to fully demonstrate if replacement therapy with reg I may prove useful in patients with impaired glucose tolerance secondary to chronic pancreatitis and maybe even with diabetes associated with aging.

Since the pathophysiology of glucose intolerance is similar in rats and in humans, and further since the Reg proteins are similar in rats and humans, the present invention is applicable to humans as well as rats. The human Reg will provide a similar function in humans as the rat Reg provides in rats.

The following table (Table 1) sets forth the sequence listings used in the present invention:

TABLE 1

| | | |
|---|---|---|
| Seq. 1 | ctg gcctctctgattaaggag | primer |
| | | up for |
| | | base |
| | | pair |
| | | probe |
| Seq. 2 | tcagatgattt caggctttaa | primer |
| | | down |
| | | for |
| | | base |
| | | pair |
| | | probe |
| Seq. 3 | tacagctgccaatgtctggatt | primer |
| | | forward |
| Seq. 4 | cagtgtcccaggatttgtagaga | primer |
| | | reverse |
| Seq. 5 | atcccaaaaataatcgccgctggc-ta | probe |
| Seq. 6 | agcagaattccaggaggctgaa gaagatctac | forward |
| | | primer |
| Seq. 7 | ctcactcgagt | reverse |
| | caggctttgaacttgcagacaaatgataattggg | primer |
| | catc | |
| Seq. 8 | ttgtcca gaaggttccaatg | forward |
| | | primer |
| Seq. 9 | caaactcaggata caagaaa | reverse |
| | | primer |
| Seq. 10 | ccccccccaa cagacttttg tctcagcctg | rat gene |
| | cagagattgt tgacttgcat cctaagcaga | coding |
| | agacagtctg ctgctcatca tgactcgcaa | sequence |
| | caaatatttc attctgcttt catgcctgat | DNA |
| | ggtcctttct ccaagccaag gccaggaggc | |
| | tgaagaagat ctaccatctg ccaggatcac | |
| | ttgtccagaa ggttccaatg cctacagttc | |
| | ctactgttac tacttcatgg aagaccattt | |
| | atcttgggct gaggcagatc ttttttgcca | |
| | gaacatgaat tcaggctact tggtgtcagt | |
| | gctcagccag gctgagggca actttctggc | |
| | ctctctgatt aaggagagtg gtactacagc | |
| | tgccaatgtc tggattggcc tccatgatcc | |
| | caaaaataat cgccgctggc actggagcag | |
| | tgggtctctg tttctctaca aatcctggga | |
| | cactgggtat cctaacaatt ccaatcgtgg | |
| | ctactgtgta tctgtgactt caaactcagg | |
| | atacaagaaa tggagagata acagttgtga | |
| | tgcccaatta tcatttgtct gcaagttcaa | |
| | agcctgaaat catctgaaaa aaatagtcat | |
| | acagagccag acaagaaaat actatggagt | |
| | caaaagtgaa actagaccat ctatcaaaag | |
| | caaagtcaac cccctcttcc tagacaaaca | |
| | ttcttgcctc actgccctat ggtattttta | |
| | tctccattat tgtatgtaac cctgcacatt | |
| | taaataaaaa taccttcaca ataaaa | |
| Seq. 11 | MTRNKYFILLSCLMVLSPSQGQEAEEDLPSARIT | protein |
| | CPEGSNAYSSYCYYFMEDHLSWAEADLFCQNMNS | coding |
| | GYLVSVLSQAEGNFLASLIKESGTTAANVWIGLH | for rat |
| | DPKNNRRWHWSSGSLFLYKSWDTGYPNNSNRGYC | |
| | VSVTSNSGYKKWRDNSCDAQLSFV CKFKA | |

### REFERENCES

The following references, which provide background as to various methods used in the present application, are incorporated by reference in their entireties herein:
1. Shamburek R D, Scott R B, Farrar J T. Gastrointestinal and liver changes in the elderly. In: Katlic M R, ed. Geriatric Surgery: Comprehensive Care of the Elderly Patient. Baltimore, Md.: Urban and Schwarzenberg; 1990: 97-115.
2. Owyang C. Endocrine changes in pancreatic insufficiency. In: Go V L W, Dimangno E P, Gardner J D, et al., eds. The Pancreas: Biology, Pathobiology and Disease. 2nd ed. New York, N.Y.: Raven Press; 1993: 803-813.
3. Centers for Disease Control and Prevention. National Diabetes Fact Sheet: General Information and National Estimates on Diabetes in the United States. Atlanta, Ga.: US Dept of Health and Human Services; 2005.
4. Zenilman M E, Magnuson T H, Swinson K, et al. Pancreatic thread protein is mitogenic to pancreatic-derived cells in culture. Gastroenterology. 1996; 110:1208-1214.
5. Levine J L, Patel K J, Zheng Q, et al. A recombinant rat regenerating protein is mitogenic to pancreatic derived cells. J Surg Res. 2005; 89:60-65.
6. Bluth M H, Patel S A, Dieckgraefe B K, et al. Pancreatic regenerating protein (reg I) and reg I receptor mRNA are upregulated in rat pancreas after induction of acute pancreatitis. World J. Gastroenterol. 2006; 12:4511-4516.
7. Zenilman M E, Chen J, Magnuson T H. Effect of reg protein on rat pancreatic ductal cells. Pancreas. 1998; 17:256-261.
8. Watanabe T, Yonemura T, Yonekura H, et al. Pancreatic beta-cell replication and amelioration of surgical diabetes by reg protein. Proc Natl Acad Sci USA. 1994; 91:3589-3592.
9. Gross D J, Weiss L, Reibstein I, et al. Amelioration of diabetes in nonobese diabetic mice with advanced disease by linomide-induced immunoregulation combined with reg protein treatment. Endocrinology. 1998; 139:2369-2374.
10. Kobayashi S, Akiyama T, Nata K, et al. Identification of a receptor for reg (regenerating gene) protein, a pancreatic beta-cell regeneration factor. J Biol. Chem. 2000; 275:10723-10726.
11. Sanchez D, Gmyr V, Kerr-Conte J, et al. Implication of Reg I in human pancreatic duct-like cells in vivo in the pathological pancreas and in vitro during exocrine dedifferentiation. Pancreas. 2004; 29:14-21.
12. Boquist L, Edstrom C. Ultrastructure of pancreatic acinar and islet parenchyma at various intervals after duct ligation. Virchows Arch A Pathol Pathol Anat. 1970; 349:69-79.
13. Edstrom C, Falkmer S. Qualitative and quantitative morphology of the rat pancreatic islet tissue five weeks after ligation of the pancreatic ducts. Acta Soc Med. Ups. 1967; 72:376-390.
14. Hultquist G T, Karlsson U, Hallner A C. The regenerative capacity of the pancreas in duct-ligated rats. Exp Pathol (Jena). 1979; 17:44-52.
15. Edstrom C, Falkmer S. Pancreatic morphology and blood glucose levels in rats at various intervals after duct ligation. Virchows Arch Abt A Path Anat. 1968; 345:139-153.
16. Hultquist G T, Jonsson L E. Ligation of the pancreatic duct in rats. Acta Soc Med. Ups. 1965; 70:82-88.
17. Edstrom C. Glucose tolerance of rats at various intervals following ligation of the pancreatic ducts. Acta Soc Med. Ups. 1971; 76:39-48.
18. Kandil E, Lin Y Y, Bluth M H, et al. Dexamethasone mediates protection against acute pancreatitis via upregulation of pancreatitis-associated proteins. World J. Gastroenterol. 2006; 12:6806-6811.
19. Zenilman M E, Tuchman D, Zheng Qh, et al. Comparison of pancreatic reg I and reg III levels in acute pancreatitis. Ann Surg. 2000; 232:646-652.
20. Tezel E, Nagasaka T, Tezel G, et al. REG I as a marker for human pancreatic acinoductular cells. Hepatogastroenterology. 2004; 51:91-96.
21. Unno M, Yonekura H, Nakagawara K, et al. Structure, chromosomal localization, and expression of mouse reg genes, reg I and reg II. A novel type of reg gene, reg II, exists in the mouse genome. J Biol. Chem. 1993; 268:15974-15982.
22. Zhang H, Kandil E, Lin Y Y, et al. Targeted inhibition of gene expression of pancreatitis-associated proteins exacerbates the severity of acute pancreatitis in rats. Scand J. Gastroenterol. 2004; 39:870-881.
23. Pierre J, Mueller C M, Viterbo D, et al. Pancreatic regeneration protein (Reg) modulates glucose tolerance testing. J Surg Res. 2007; 131:313.
24. Idezuki Y, Goetz F C, Lillehei R C. Late effect of pancreatic duct ligation on β-cell function. Am J. Surg. 1969; 117:33-39.
25. Kloppel G, Bommer G, Commandeur G, et al. The endocrine pancreas in chronic pancreatitis. Immunocytochemical and ultrastructural studies. Virchows Arch A Pathol Anat Histol. 1978; 377:157-174.
26. Rosenberg L, Duguid W P, Brown R A, et al. Induction of nesidioblastosis will reverse diabetes in Syrian golden hamster. Diabetes. 1988; 37: 334-341.
27. Lillemoe K D. Pancreatic disease in the elderly patient. Surg Clin North Am. 1994; 74:317-344.
28. Hellerstrom C. The life story of the pancreatic β-cell. Diabetologia. 1984; 26:393-400.
29. Dragstedt L R. Some physiologic problems in surgery of the pancreas. Ann Surg. 1943; 118:576-593.
30. Yeo C J, Bastidas J A, Schmeig R E, et al. Pancreatic structure and glucose tolerance in a longitudinal study of experimental pancreatitis-induced diabetes. Ann Surg. 1989; 210:150-158.
31. Gooszen H G, Schilfgaarde R V, van der Burg M P M, et al. Quantitative assessment of long-term changes in insulin secretion after canine duct-obliterated pancreas transplantation. Transplantation. 1988; 46: 793-799.
32. Perfetti R, Raydada M, Wang Y, et al. Reg and insulin genes are expressed in prepancreatic mouse embryos. J Mol. Endocrinol. 1996; 17:1-10.
33. Unno M, Itoh T, Watanabe T, et al. Islet beta-cell regeneration and reg genes. Adv Exp Med. Biol. 1992; 321:61-69.
34. Zenilman M E, Perfetti R, Swinson K, et al. Pancreatic regeneration (reg) gene expression in a rat model of islet hyperplasia. Surgery. 1996; 119:576-584.
35. Planas R, Alba A, Carrillo J, et al. Reg (regenerating) gene overexpression in islets from non-obese diabetic mice with accelerated diabetes: role of IFNbeta. Diabetologia. 2006; 49:2379-2387.
36. Baeza N, Sanchez D, Vialettes B, et al. Specific reg II gene overexpression in the non-obese diabetic mouse pancreas during active diabetogenesis. FEBS Lett. 1997; 416:364-368.
37. Shervani N J, Takasawa S, Uchigata Y, et al. Autoantibodies to REG, a beta-cell regeneration factor, in diabetic patients. Eur J Clin Invest. 2004; 34:752-758.
38. Unno M, Nata K, Noguchi N, et al. Production and characterization of Reg knockout mice: reduced proliferation of pancreatic beta-cells in Reg knockout mice. Diabetes. 2002; 51:S478-S483.
39. Yamaoka T, Yoshino K, Yamada T, et al. Diabetes and tumor formation in transgenic mice expressing Reg I. Biochem Biophys Res Commun. 2000; 278:368-376.
40. Zenilman M E, Zheng Q, Wu H, et al. Pancreatic reg and a conserved bioactive fragment are mitogenic through the MAPK p38 pathway. Surg Forum. 2000;LI:330-34.
41. Wu H, Fan Z, Patel, et al. Expression of reg receptor in rat pancreatic cell lines. Gastroenterology. 2001; 120:A338.
42. Okamoto H, Takasawa S. Recent advances in the Okamoto model: the CD38-cyclic ADP-ribose signal system and the regenerating gene protein (Reg)-Reg receptor system in beta-cells. Diabetes. 2002; 51:S462-S473.
43. Rafaeloff R, Pittenger G L, Barlow S W, et al. Cloning and sequencing of the pancreatic islet neogenesis associated protein (INGAP) gene and its expression in islet neogenesis in hamsters. J Clin Invest. 1997; 99: 2100-2109.
44. Taylor-Fishwick D A, Rittman S, Kendall H et al. Cloning genomic INGAP: a Reg-related family member with distinct transcriptional regulation sites. Biochim Biophys Acta. 2003; 1638:83-89.
45. Rosenberg L, Lipsett M, Yoon J W, et al. A pentadecapeptide fragment of islet neogenesis-associated protein increases beta-cell mass and reverses diabetes in C57BL/6J mice. Ann Surg. 2004; 240:875-884.
46. Pierre J, Mueller C M, Bernstein E, et al. The effect of REG administration on glycosylated hemoglobin in aged rats. J Surg Res. 2008; 144: 376.

What is claimed is:

1. A method of treating diabetes, comprising:
administering to a mammal diagnosed with diabetes a purified recombinant reg I protein.

2. The method of claim 1, further wherein the mammal is diagnosed with diabetes.

3. The method of claim 1, further wherein the mammal is diagnosed with pancreatitis.

4. The method of claim 1, further wherein the mammal is diagnosed with a low glucose tolerance.

5. The method of claim 1, further comprising one or more steps, selected from the steps including: measuring a result;
correlating a result against a standard;
observing a result;
co-administering at least one therapeutic agent with said purified recombinant reg;
repeating the administering step; and
combinations thereof.

6. A purified recombinant pancreatic regeneration protein I (recombinant reg I), comprising the (full rat gene coding sequence) sequence ID:
ORIGIN

| [Seq. ID No. 10] | |
|---|---|
| 1 | ccccccccaa cagacttttg tctcagcctg cagagattgt tgacttgcat cctaagcaga |
| 61 | agacagtctg ctgctcatca tgactcgcaa caaatatttc attctgcttt catgcctgat |
| 121 | ggtcctttct ccaagccaag gccaggaggc tgaagaagat ctaccatctg ccaggatcac |
| 181 | ttgtccagaa ggttccaatg cctacagttc ctactgttac tacttcatgg aagaccattt |
| 241 | atcttgggct gaggcagatc ttttttgcca gaacatgaat tcaggctact tggtgtcagt |
| 301 | gctcagccag gctgagggca actttctggc ctctctgatt aaggagagtg gtactacagc |
| 361 | tgccaatgtc tggattggcc tccatgatcc caaaaataat cgccgctggc actggagcag |
| 421 | tgggtctctg tttctctaca aatcctggga cactgggtat cctaacaatt ccaatcgtgg |
| 481 | ctactgtgta tctgtgactt caaactcagg atacaagaaa tggagagata acagttgtga |
| 541 | tgcccaatta tcatttgtct gcaagttcaa agcctgaaat catctgaaaa aaatagtcat |
| 601 | acagagccag acaagaaaat actatggagt caaaagtgaa actagaccat ctatcaaaag |
| 661 | caaagtcaac cccctcttcc tagacaaaca ttcttgcctc actgccctat ggtattttta |
| 721 | tctccattat tgtatgtaac cctgcacatt taaataaaaa taccttcaca ataaaa; |

wherein the full protein coding data for rat reg I comprises the following sequence:
[Seq. ID No. 11]
MTRNKYFILLSCLMVLSPSQGQEAEEDLPSARITCPEGSNAYSSYCYYFM
EDHLSWAEADLFCQNMNSGYLVSVLSQAEGNFLASLIKESGTTAANVWIG
LHDPKNNRRWHWSSGSLFLYKSWDTGYPNNSNRGYCVSVTSNSGYKKWRD
NSCDAQLSFV CKFKA
at about 100% homology.

7. The recombinant reg I of claim 6, further comprising:
a delivery agent.

8. A method of treating diabetes in a mammal requiring treatment thereof, comprising administering replacement therapy to replace reg I.

9. The method of claim 8, wherein the administering step further includes administering a recombinant reg I in a substantially pure form and a pharmaceutically acceptable carrier.

10. A method of making recombinant pancreatic regeneration protein I, comprising:
synthesizing the recombinant reg I protein;
replicating the recombinant reg I protein;
isolating the recombinant reg I protein; and
purifying the recombinant reg I protein.

11. A method of producing purified recombinant reg I, comprising:
producing a plurality of recombinant rat His-tagged reg I protein in a plurality of E. coli by EcoRI-Xho I directional cloning;
administering a plurality of Xho/EcoRI restriction enzymes to amplify and digest a plurality of full-length reg I product;
inserting a plurality of digested reg I PCR amplicons in-frame into a pET24a bacterial expression vector to positively clone the reg I PCR amplicons;
transforming the reg I PCR amplicons into a plurality of BL21 (DE3) E. coli to promote growth;
removing at least one soluble bacterial protein from the E. coli bacteria to obtain a bacterial pellet;
washing and centrifuging the bacterial pellet to form a second bacterial pellet;
resolubilizing the second bacterial pellet in a second resuspension buffer at a low temperature;
collecting the solubilized proteins onto a plurality of gel-beads;
spinning and washing the gel beads at least once with a wash buffer followed by centrifuge;
administering an elution buffer to the gel-beads; and
dialyzing a reg I protein abundant elution in a dialysis buffer to enhance refolding and to prevent precipitation of a purified reg I protein.

12. The method of claim 11, further wherein the producing step further comprises using forward primer: 5′-AGCAGAATTCCAGGAGGCTGAA GAAGATCTAC-3′ [Seq. ID No. 6] and reverse primer: 5′-CTCACTCGAGTCAGGCTTTGAACTTGCAGACAAATGATA ATTGGG CATC-3′ [Seq. ID No. 7].

13. The method of claim 11, further comprising the step of confirming the reg I-containing constructs by PCR (forward: 5′-TTGTCCA GAAGGTTCCAATG-3′ [Seq. ID No. 8], reverse: 5′-CAAACTCAGGATA CAAGAAA-3′ [Seq. ID No. 9]).

14. The method of claim 11, further wherein the transforming step further comprises growing the reg I PCR amplicons to a desired density.

15. The method of claim 11, wherein the removing step further includes centrifuging and resuspending the E. coli in a resuspension buffer containing a protease inhibitor and further sonicating said resuspension buffer and the E. coli at a low temperature.

16. The method of claim 11, wherein the collecting step further comprises centrifuging the solubilized proteins at a low temperature followed by batch binding the proteins to a plurality of prepared His-Select Nickel Affinity Gel beads.
